(19) United States
(12) Patent Application Publication
(10) Pub. No.: US 2018/0318551 A1
(43) Pub. Date: Nov. 8, 2018

(54) CATHETER WITH IMPROVED TORQUE RESPONSE

(71) Applicant: BOSTON SCIENTIFIC SCIMED, INC., MAPLE GROVE, MN (US)

(72) Inventors: JOEL M. WASDYKE, EDEN PRAIRIE, MN (US); SIMRANJEET BHAGAT, PATHANKOT (IN)

(73) Assignee: BOSTON SCIENTIFIC SCIMED, INC., MAPLE GROVE, MN (US)

(21) Appl. No.: 15/970,561

(22) Filed: May 3, 2018

Related U.S. Application Data
(60) Provisional application No. 62/502,335, filed on May 5, 2017.

(51) Int. Cl. A61M 25/00

(52) U.S. Cl. CPC A61M 25/0053; A61M 25/09; A61M 25/0052

(57) ABSTRACT

Medical devices and methods for making and using medical devices are disclosed. An example medical device may include a catheter. The catheter may include an elongate shaft having a proximal end, a distal end, and a lumen extending at least partially between the proximal end and the distal end. The shaft may include an inner layer, a reinforcing member, and an outer layer. The outer layer may include a first section positioned adjacent to the proximal end, a second section disposed distally of the first section, a third section positioned distally of the second section, and a fourth section positioned distally of the third section.

### CROSS-REFERENCE TO RELATED APPLICATIONS

This application claims the benefit of priority under 35 U.S.C. § 119 to U.S. Provisional Application Ser. No. 62/502,335, filed May 5, 2017, the entirety of which is incorporated herein by reference.

### TECHNICAL FIELD

The present disclosure pertains to medical devices, and methods for manufacturing medical devices. More particularly, the present disclosure pertains to elongated intracorporeal medical devices with an improved torque response.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example catheter is disclosed. The catheter comprises: an elongate shaft having a proximal end, a distal end, and a lumen extending at least partially between the proximal end and the distal end; wherein the outer layer includes a first section positioned adjacent to the proximal end, a second section disposed distally of the first section, a third section positioned distally of the second section, and a fourth section positioned distally of the third section; wherein the first section has a first flexural rigidity; wherein the second section has a second flexural rigidity that is lower than the first flexural rigidity; wherein the third section has a third flexural rigidity that is higher than the second flexural rigidity; and wherein the fourth section has a fourth flexural rigidity that is lower than the third flexural rigidity.

Alternatively or additionally to any of the embodiments above, wherein the shaft includes an inner layer and wherein the inner layer includes polytetrafluoroethylene.

Alternatively or additionally to any of the embodiments above, wherein the shaft includes an inner layer and wherein the inner layer comprises a coextrusion of polytetrafluoroethylene and a polyether block amide.

Alternatively or additionally to any of the embodiments above, wherein the shaft includes a reinforcing member and wherein the reinforcing member includes a braid.

Alternatively or additionally to any of the embodiments above, the first flexural rigidity and the third flexural rigidity are substantially the same.

Alternatively or additionally to any of the embodiments above, the second flexural rigidity and the fourth flexural rigidity are substantially the same.

Alternatively or additionally to any of the embodiments above, the first section, the third section, or both include a nylon.

Alternatively or additionally to any of the embodiments above, the second section, the fourth section, or both include a polyether block amide.

Alternatively or additionally to any of the embodiments above, the first section has a first length, wherein the second section has a second length, wherein the third section has a third length, wherein the fourth section has a fourth length, and wherein the fourth length is shorter than at least one of the first length, the second length, or the third length.

An example catheter is disclosed. The catheter comprises: an inner layer defining a guidewire lumen; a reinforcing member disposed along the inner layer; an outer layer disposed along the reinforcing member; wherein the outer layer includes a first section, a second section disposed distally of the first section, a third section positioned distally of the second section, and a fourth section positioned distally of the third section; wherein the first section has a first flexural rigidity; wherein the second section has a second flexural rigidity that is lower than the first flexural rigidity; wherein the third section has a third flexural rigidity that is higher than the second flexural rigidity; and wherein the fourth section has a fourth flexural rigidity that is lower than the third flexural rigidity.

Alternatively or additionally to any of the embodiments above, the inner layer comprises a coextrusion of polytetrafluoroethylene and a polyether block amide.

Alternatively or additionally to any of the embodiments above, the reinforcing member includes a braid.

Alternatively or additionally to any of the embodiments above, the first flexural rigidity and the third flexural rigidity are substantially the same, wherein the second flexural rigidity and the fourth flexural rigidity are substantially the same, or both.

Alternatively or additionally to any of the embodiments above, the first section, the third section, or both include a nylon.

Alternatively or additionally to any of the embodiments above, the second section, the fourth section, or both include a polyether block amide.

Alternatively or additionally to any of the embodiments above, the first section has a first length, wherein the second section has a second length, wherein the third section has a third length, wherein the fourth section has a fourth length, and wherein the fourth length is shorter than at least one of the first length, the second length, or the third length.

A method for accessing an intravascular target region is disclosed. The method comprises: advancing a catheter through a blood vessel, the catheter comprising: an elongate shaft having a proximal end, a distal end, and a lumen extending at least partially between the proximal end and the distal end, wherein the shaft includes an inner layer, a reinforcing layer, and an outer layer, wherein the outer layer includes a first section positioned adjacent to the proximal end, a second section disposed distally of the first section, a third section positioned distally of the second section, and a fourth section positioned distally of the third section, wherein the first section has a first flexural rigidity, wherein the second section has a second flexural rigidity that is lower than the first flexural rigidity, wherein the third section has a third flexural rigidity that is higher than the second flexural rigidity, and wherein the fourth section has a fourth flexural rigidity that is lower than the third flexural rigidity.

Alternatively or additionally to any of the embodiments above, advancing a catheter through a blood vessel includes positioning the section across an arterial bifurcation.

Alternatively or additionally to any of the embodiments above, the arterial bifurcation includes an iliac bifurcation.

Alternatively or additionally to any of the embodiments above, advancing a catheter through a blood vessel includes positioning the fourth section within an artery below a knee of a patient.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a cross-sectional view of an example medical device.

FIG. 2 schematically illustrates the use of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A number of medical devices may be used to access different parts of the anatomy. For example, some catheters can be used to access blood vessels relatively deep within the anatomy. When a catheter contacts curves, bends, tight radii, or the like, the ability of the catheter to transmit rotational forces and/or torque applied at the proximal end of the catheter to the distal end of the catheter may be reduced. Disclosed herein are medical devices such as catheters that are designed to transmit rotational forces and/or torque applied at the proximal end of the catheter to the distal end of the catheter.

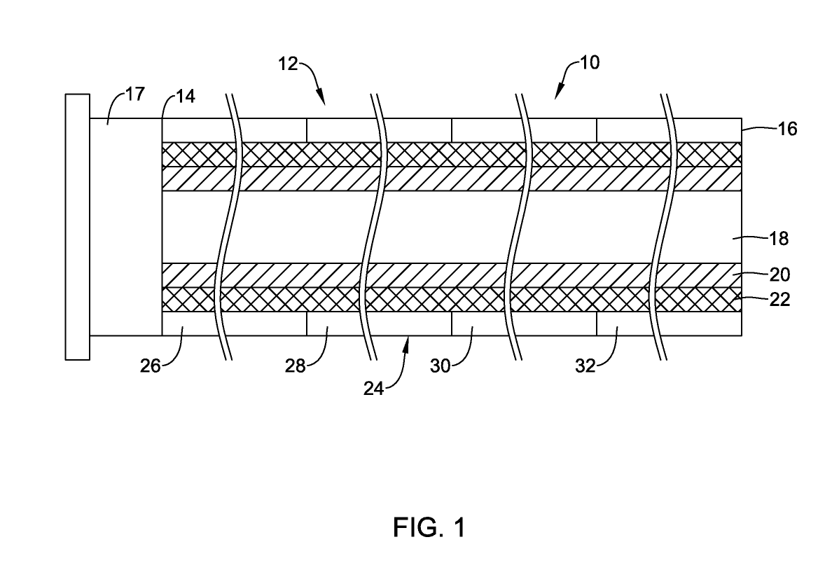

FIG. 1 illustrates an example catheter 10. The catheter 10 includes a catheter shaft 12 having a proximal end 14, a distal end 16, and a lumen 18 extending at least partially the length there through. In at least some instances, the lumen 18 may have a diameter on the order of about 0.005 to 0.05 inches or about 0.014 to about 0.040 inches. For example, the lumen 18 may be designed to accommodate a 0.014-0.018 inch guidewire. In some of these and in other instances, the lumen 18 may be designed to accommodate a 0.035 inch or 0.038 inch guidewire. Other sizes and/or dimensions are contemplated. A hub 17 may be coupled to the proximal end 14 of the catheter shaft 12.

The catheter shaft 12 may include an inner layer 20. The inner layer 20 may include a lubricous material such as polytetrafluoroethylene, fluorinated ethylene propylene, or the like. In some instances, the inner layer 20 may be formed as a coextrusion of polytetrafluoroethylene and a polyether block amide. In some instances, the inner layer 20 may be formed as a coextrusion of polytetrafluoroethylene and another material such as any suitable one(s) of those materials disclosed herein. In some instances, the inner layer 20 may be formed as a continuous extrusion of PEBAX series of nylon polymers with or without added radiopaque fillers like bismuth subcarbonate. In still other instances, the inner layer may be formed from or otherwise include a polyether block amide (e.g., PEBAX), a nylon (e.g., VESTAMID), or the like. Other materials are contemplated. In some instances, the inner layer 20 extends along substantially the entire length of the catheter shaft 12. For example, the distal end of the inner layer 20 may be axially-aligned with the distal end 16 of the catheter shaft 12, the proximal end of the inner layer 20 may be axially-aligned with the proximal end 14 of the catheter shaft 12, or both. Alternatively, the inner layer 20 may extend distally beyond the distal end 16 of the catheter shaft 12, the inner layer 20 may extend proximally beyond the proximal end 14 of the catheter shaft 12, or both. In some instances, the inner layer 20 may extend along only a portion of the length of the catheter shaft 12. In some instances, the inner layer 20 is fixedly attached to other portions of the catheter shaft 12. Alternatively, portions or all of the inner layer 20 may be free from attachment with other portions of the catheter shaft 12. In some instances, the inner layer 20 may be single, continuous monolith of material. Alternatively, the inner layer 20 include a plurality of discrete sections. The discrete sections, which may or may not be formed from the same material, may abut or overlap with one another. Alternatively, some or all of the sections may be axially spaced from one another.

A reinforcing member 22 may extend along at least a portion of the inner layer 20. The reinforcing member 22 may include a braid, coil, mesh, or the like. For example, the reinforcing member 22 may include a stainless steel braid. Other materials are contemplated including those materials disclosed herein. The reinforcing member 22 may be positioned along the outer surface of the inner layer 20. Alternatively, the reinforcing member 22 may be partially disposed within or completely embedded within the inner layer 20 (and/or other portions of the catheter shaft 12 including, for example, an outer layer 24 of the catheter shaft, which is discussed herein). The reinforcing member 22 may extend along substantially the entire length of the catheter shaft 12 or along only a portion of the catheter shaft 12. In some instances, a plurality of discrete reinforcing members 22 may be utilized.

An outer layer 24 may extend along at least a portion of the reinforcing member 22. In some instances, the outer layer 24 may be single, continuous monolith of material. Alternatively, the outer layer 24 include a plurality of discrete sections. For example, the outer layer 24 may include a first section 26, a second section 28 disposed distally of the first section 26, a third section 30 disposed distally of the second section 28, and a fourth section 32 disposed distally of the third section 30. The sections 26/28/30/32 may abut or overlap with one another. Alternatively, some or all of the sections 26/28/30/32 may be axially spaced from one another. In some instances, all of the sections 26/28/30/32 are formed from the same material. Alternatively, at least some of the sections 26/28/30/32 are formed from different materials.

In at least some instances, the outer layer 24 may be designed to provide the catheter shaft 12 (and/or the catheter 10, in general) with a number of desirable characteristics. For example, when navigating a medical device through the tortuous anatomy (e.g., including accessing relatively small vessels below-the-knee), contact with the vessel wall along relative tight curves may reduce the ability of the device to transmit the rotation being input at the proximal end of the device. The catheter 10 is designed to have an improved torque response so that rotational forces applied at the proximal end 14 of the catheter shaft 12 can be efficiently transmitted to the distal end 16 (and/or toward the distal end 16). This may include efficiently transmitting rotational forces while the catheter shaft 12 is positioned along a relatively tight curve in the vasculature.

In order to provide catheter 10 (and/or catheter shaft 12) with the desired torque response/responsiveness, the outer layer 24 may be designed so that the sections 26/28/30/32 provide the desired level of flexibility/stiffness along the length of the catheter shaft 12. For example, the first section 26, which may be positioned adjacent to the proximal end 14 of the catheter shaft 12, may have a first flexural rigidity. The first flexural rigidity may understood as being relatively stiff. The second section 28 may have a second flexural rigidity. In at least some instances, the second flexural rigidity is less than the first flexural rigidity. The third section 30 may have a third flexural rigidity. In at least some instances, the third flexural rigidity is greater than the second flexural rigidity. The fourth section 32 may have a fourth flexural rigidity. In at least some instances, the fourth flexural rigidity is less than the third flexural rigidity. In some instances, the first flexural rigidity may be substantially the same as the third flexural rigidity. In some of these and in other instances, the second flexural rigidity may be substantially the same as the fourth flexural rigidity.

In some instances, the sections 26/28/30/32 of the outer layer 24 may include materials that may help provide the desired flexibility/stiffness characteristics (e.g., flexural rigidity) and/or torque response. For example, the first section 26 may include a polymeric material such as a nylon. One example material that may be utilized for first section 26 is VESTAMID L2101. Other materials may be used including those materials disclosed herein. The second section 28 may include a polymeric material such as a polyether block amide. One example material that may be utilized for second section 28 is a 55D PEBAX or 63D PEBAX. Other materials may be used including those materials disclosed herein. The third section 30 may include a polymeric material such as a nylon. One example material that may be utilized for third section 30 is a VESTAMID L2101. Other materials may be used including those materials disclosed herein. The fourth section 32 may include a polymeric material such as a polyether block amide. One example material that may be utilized for fourth section 32 is a 55D PEBAX or 63D PEBAX. More or fewer than four sections may be utilized, depending on the intended use. In general, the softer segments may be disposed along sections of the catheter that align with or are otherwise subject to stay in tighter curve while rotation of the catheter 10 is needed.

The sections 26/28/30/32 of the outer layer 24 may have lengths that are designed to aid the use of the catheter 10. For example, the first section 26 may have a length on the order of about 20-80 cm, or about 30-50 cm, or about 40 cm. The second section 28 may have a length that is the same as or different from the first section 26 (and/or other sections). For example, the second section 28 may have length on the order of about 20-80 cm, or about 30-50 cm, or about 40 cm. The third section 30 may have a length that is the same as or different from the second section 28 (and/or other sections). For example, the third section 30 may have length on the order of about 20-80 cm, or about 30-50 cm, or about 40 cm. The fourth section 32 may have a length that is the same as or different from the third section 30 (and/or other sections). For example, the fourth section 32 may have length on the order of about 15-75 cm, or about 20-40 cm, or about 30 cm. In at least some instances, the fourth section 32 may have a length that is shorter than the first section 26, the second section 28, the third section 30, or combinations thereof.

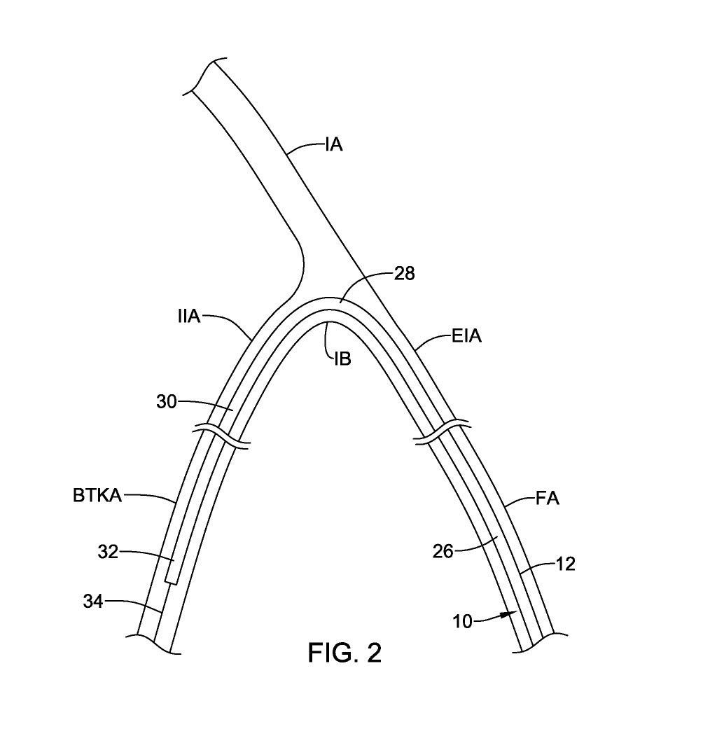

In general, the design of the outer layer 24 may be tailored so that when the catheter 10 is used to access vascular locations where spanning a tight curve may be necessary, the first section 26 may be positioned along a generally straight and wide portion of the vasculature, the second section 28 may be positioned across the tight curve, the third section 30 may be positioned along a less tortuous portion of vasculature, and the fourth section 32 may be positioned in a target region, which may be relatively deep within the anatomy. For example, FIG. 2 schematically illustrate an example use of the catheter 10. In this example, the femoral artery FA, the iliac artery IA, the iliac bifurcation IB, the external iliac artery EIA, the internal iliac artery IIA, and a below-the-knee artery BTKA are schematically shown. The catheter 10 may be navigated, for example over a guidewire 34, through the femoral artery FA, into the external iliac artery IEA, and to a position adjacent to the iliac bifurcation IB. The catheter 10 may be further advanced through the iliac bifurcation IB, through the internal iliac artery IIA, and to a position within the below-the-knee artery BTKA. The design of the outer layer 24 may be arranged so that the first section 26 may extend along the femoral artery FA and/or along a portion of the external iliac artery IEA, the second section 28 may span or otherwise be disposed across the iliac bifurcation IB, the third section 30 may be positioned along the internal iliac artery IIA, and the fourth section 32 may be positioned within a relatively small below-the-knee artery BTKA (e.g., such as the anterior tibial artery, the posterior tibial artery, the peroneal artery, etc.).

The method for manufacturing the catheter 10 may include a number of processes. For example, the inner layer 20 may be disposed along a mandrel. This may include mechanically disposing the inner layer 20 onto the mandrel, extrusion, dip coating, spray coating, or another suitable process. The reinforcing member 22 may be disposed along the inner layer 20. The sections 26/28/30/32 may be disposed along the reinforcing member 22. In some instances, a heat shrink tube may be disposed over the sections 26/28/30/32 and heat may be applied. The heat may cause the sections 26/28/30/32 to reflow and bond with one another and bond with the reinforcing member 22 and/or inner layer 20.

The materials that can be used for the various components of the catheter 10 may include those commonly associated with medical devices. For simplicity purposes, the following discussion makes reference to the catheter shaft 12 and other components of the catheter 10. However, this is not intended to limit the devices and methods described herein, as the discussion may be applied to other similar shafts and/or components of the catheter 10.

The catheter shaft 12 and/or other components of the catheter 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon, VESTAMID® materials such as VESTAMID® L2101 available from EVONIK), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the catheter 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the catheter 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter 10 to achieve the same result.

In some embodiments, a degree of Magnetic Resonance Imaging (MRI) compatibility is imparted into the catheter 10. For example, the catheter 10, or portions thereof, may be made of a material that does not substantially distort the image and create substantial artifacts (e.g., gaps in the image). Certain ferromagnetic materials, for example, may not be suitable because they may create artifacts in an MRI image. The catheter 10, or portions thereof, may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A catheter, comprising:
an elongate shaft having a proximal end, a distal end, and a lumen extending at least partially between the proximal end and the distal end;
wherein the outer layer includes a first section positioned adjacent to the proximal end, a second section disposed distally of the first section, a third section positioned distally of the second section, and a fourth section positioned distally of the third section;
wherein the first section has a first flexural rigidity;
wherein the second section has a second flexural rigidity that is lower than the first flexural rigidity;
wherein the third section has a third flexural rigidity that is higher than the second flexural rigidity; and
wherein the fourth section has a fourth flexural rigidity that is lower than the third flexural rigidity.

2. The catheter of claim 1, wherein the shaft includes an inner layer and wherein the inner layer includes polytetrafluoroethylene.

3. The catheter of claim 1, wherein the shaft includes an inner layer and wherein the inner layer comprises a coextrusion of polytetrafluoroethylene and a polyether block amide.

4. The catheter of claim 1, wherein the shaft includes a reinforcing member and wherein the reinforcing member includes a braid.

5. The catheter of claim 1, wherein the first flexural rigidity and the third flexural rigidity are substantially the same.

6. The catheter of claim 1, wherein the second flexural rigidity and the fourth flexural rigidity are substantially the same.

7. The catheter of claim 1, wherein the first section, the third section, or both include a nylon.

8. The catheter of claim 1, wherein the second section, the fourth section, or both include a polyether block amide.

9. The catheter of claim 1, wherein the first section has a first length, wherein the second section has a second length, wherein the third section has a third length, wherein the fourth section has a fourth length, and wherein the fourth length is shorter than at least one of the first length, the second length, or the third length.

10. A catheter shaft, comprising:
an inner layer defining a guidewire lumen;
a reinforcing member disposed along the inner layer;
an outer layer disposed along the reinforcing member;
wherein the outer layer includes a first section, a second section disposed distally of the first section, a third section positioned distally of the second section, and a fourth section positioned distally of the third section;
wherein the first section has a first flexural rigidity;
wherein the second section has a second flexural rigidity that is lower than the first flexural rigidity;
wherein the third section has a third flexural rigidity that is higher than the second flexural rigidity; and
wherein the fourth section has a fourth flexural rigidity that is lower than the third flexural rigidity.

11. The catheter shaft of claim 10, wherein the inner layer comprises a coextrusion of polytetrafluoroethylene and a polyether block amide.

12. The catheter shaft of claim 10, wherein the reinforcing member includes a braid.

13. The catheter shaft of claim 10, wherein the first flexural rigidity and the third flexural rigidity are substantially the same, wherein the second flexural rigidity and the fourth flexural rigidity are substantially the same, or both.

14. The catheter shaft of claim 10, wherein the first section, the third section, or both include a nylon.

15. The catheter shaft of claim 10, wherein the second section, the fourth section, or both include a polyether block amide.

16. The catheter shaft of claim 10, wherein the first section has a first length, wherein the second section has a second length, wherein the third section has a third length, wherein the fourth section has a fourth length, and wherein the fourth length is shorter than at least one of the first length, the second length, or the third length.

17. A method for accessing an intravascular target region, the method comprising:
advancing a catheter through a blood vessel, the catheter comprising:
an elongate shaft having a proximal end, a distal end, and a lumen extending at least partially between the proximal end and the distal end, wherein the shaft includes an inner layer, a reinforcing layer, and an outer layer, wherein the outer layer includes a first section positioned adjacent to the proximal end, a second section disposed distally of the first section, a third section positioned distally of the second section, and a fourth section positioned distally of the third section, wherein the first section has a first flexural rigidity, wherein the second section has a second flexural rigidity that is lower than the first flexural rigidity, wherein the third section has a third flexural rigidity that is higher than the second flexural rigidity, and wherein the fourth section has a fourth flexural rigidity that is lower than the third flexural rigidity.

18. The method of claim 17, wherein advancing a catheter through a blood vessel includes positioning the section across an arterial bifurcation.

19. The method of claim 18, wherein the arterial bifurcation includes an iliac bifurcation.

20. The method of claim 17, wherein advancing a catheter through a blood vessel includes positioning the fourth section within an artery below a knee of a patient.
